(19) United States
(12) Patent Application Publication
(10) Pub. No.: US 2017/0035608 A1
(43) Pub. Date: Feb. 9, 2017

(54) USING INTENSE PULSED LIGHT TO LIGHTEN EYE COLOR

(71) Applicant: Brian S. Boxer Wachler, Santa Monica, CA (US)

(72) Inventor: Brian S. Boxer Wachler, Santa Monica, CA (US)

(21) Appl. No.: 14/818,356

(22) Filed: Aug. 5, 2015

(51) Int. Cl. A61F 9/008; A61B 3/135

(52) U.S. Cl. CPC A61F 9/008; A61B 3/135; A61F 2009/00876; A61F 2009/00846

(57) ABSTRACT

The present invention relates to a device for permanently changing the color of the iris, such as from brown to blue. The invention is a system that includes a slit lamp microscope, a source of intense pulsed light (IPL), optical tracking and measuring systems, and a device that prevents the application of light to the pupil. The IPL provides a simultaneous application of a range of wavelengths, rather than the single wavelength typically applied by lasers. In a preferred embodiment, the IPL is applied as annular ring, striking only the iris and not the pupil or the sclera. Air or liquid cooling can be used to prevent the eye from overheating.

### FIELD OF THE INVENTION

This invention relates to the fields of ophthalmic and refractive surgery. It involves the use of intense pulsed light (IPL) to change eye color.

### BACKGROUND OF THE INVENTION

Eye color resides in the iris. Blue eyes, like blonde hair, are often perceived as a very desirable feature. Through evolution, however, blue and light iris colors are determined by recessive genes, which accounts for the high percentage of individuals whose eyes are a dark or brownish color that results from a dominant genetic trait. The relatively recent advent of colored contact lenses has enabled people to at least temporarily change their eye color to what is perceived as a lighter, more attractive color. Contact lenses, however, cannot permanently change eye color, and some individuals have difficulty tolerating them for even brief periods of time. Therefore, it would be desirable to have a permanent way to change dark eye colors, such as brown, to a lighter color, such as blue. For reference, FIG. 1 generally depicts features of the anterior portion of eye 12 that are discussed below. These include pupil 14, iris 15, cornea 16, sclera 18, and lens 19. Melanin in iris 15 controls the color of the eye.

A device or system capable of effecting such change would require the ability to observe and measure the patient's eye before and during such a color change procedure. This would require a slit lamp microscope. Before slit lamp illumination made a cross-sectional view of the eye possible, measuring the corneal thickness was done manually, a procedure far too imprecise for modern eye surgery. A slit lamp microscope is an ophthalmic instrument that uses a high-intensity light focused through a narrow slit to illuminate the object being viewed through the microscope's eyepiece—in this case, the patient's eyes. This kind of microscope provides a three-dimensional or stereoscopic view of the patient's eyes through a binocular eyepiece. The magnification can be adjusted using a dial located at the side of the ocular. Surgical or operating slit lamp microscopes are also equipped with a zoom function that can be operated with foot pedals so that the hands of the operator, such as a refractive surgeon, can be left free to continue working.

Providing a magnified and even a cross-sectional view of the eye, the slit lamp microscope is an important ophthalmic instrument for both ophthalmologists and eye surgeons not only for examining the eye for disorders but also for performing actual ophthalmic surgery. Examples of slit lamp microscopes can be seen in U.S. Pat. No. 4,877,321 to Ichihashi et al and U.S. Pat. No. 7,628,490 to Nakamura. FIG. 2 generally depicts a slit lamp microscope similar to that of Nakamura. It includes a base 102 mounted so that it can slide horizontally on table 103 so that the operator can focus the light on different aspects of the eye while doing the examination. Joystick 104 allows fine movement control of base 102. In addition, base 102 is rotatably mounted about axis A. Binocular microscope 105 for viewing a patient's eye is mounted on arm 107. Rotation of nob 106 permits the operator to change the magnification and viewing angle of microscope 105. Adjustable headrest frame 120 typically includes both a chin rest and a forehead rest (not shown), so that the patient's eye is aligned with the eyepieces of microscope 105. The headrest 120 also ensures minimal movement while the ocular examination (or surgery) takes place. It typically comprises metal rods. In this particular version of the prior art, mirror 110 delivers light to the patient's eye from either an illuminating lamp, a laser, or both. Mirror 110 sits atop head section 110 that controls the mirror's vertical adjustment so that it can be aligned with the patient's eye. Housing 115 contains optical elements, light sources, and other aspects necessary for the slit lamp microscope 100 to function.

The light coming from mirror 112 can be changed according to thickness and height using controls located on pivoting base 102. An operator would need to adjust the light to a broad beam and place it at full height and low brightness to look at the surface of the eye. This kind of ophthalmic examination is done under diffuse illumination. A thinner and brighter light beam of full height, on the other hand, is used for a cross-sectional view of the eye, which permits viewing the anterior segment of the eye as well as the cornea. It is called direct focal illumination.

The color of the iris gives the eye its color. The iris is a thin, circular structure in the eye, responsible for controlling the diameter and size of the pupil and thus the amount of light reaching the retina. In optical terms, the pupil is the eye's aperture and the iris is the diaphragm that serves as the aperture stop. The iris consists of two layers: the front pigmented fibrovascular region known as a stroma and, beneath the stroma, pigmented epithelial cells. The iris is usually strongly pigmented, with the color typically ranging between brown, hazel, green, gray, or blue. Despite the wide range of eye colors, the only pigment that contributes substantially to normal human iris color is the dark pigment melanin. The quantity of melanin pigment in the iris is the principal factor in determining a person's eye color. Iris color is due to variable amounts of brown-black melanins and red-yellow melanins produced by melanocytes. More brown-black melanins are found in brown-eyed people and more red-yellow melanins in blue and green-eyed people. The melanin is contained in subcellular bundles called melanosomes. It is this melanin that must be destroyed to change eye color. The peak light absorption of human melanin pigment occurs around 335 nm.

Intense pulsed light (IPL) devices are non-laser high intensity light sources that make use of a high-output flash lamp to produce a broad wavelength output of noncoherent light, usually in the 500 to 1400 nm range. The working basis of the IPL rests on the principle of selective photothermolysis, in which thermally mediated radiation damage is confined to chosen epidermal and/or dermal-pigmented targets at the cellular or tissue structural levels. IPL generated by most modern devices are produced by bursts of electrical current passing through a xenon gas-filled chamber. IPL has achieved success in resolving a variety of dermatological problems involving melanin, such as pigmented lesions of the skin. IPL systems use pulsed polychromatic light in a broad wavelength spectrum. Filters are used to allow the optimal wavelengths to penetrate the tissue. This approach contrasts with lasers, which use a single wavelength. Brown structures such as melanin absorb light in a variety of wavelengths and pulses, thus eliminating pigmented skin lesions. While popular for dermatological use, IPL has not been used for ophthalmological purposes. Equipment manufacturers warn against using IPL on the eye. A 2002 operating manual for the Lumenis Quantum SR explicitly states:
Intense pulsed light emission presents an eye hazard . . . .

Make sure that the patient and all those present in the treatment room guard against accidental exposure to this emission either directly . . . or indirectly . . . .

Never look directly at the light beam coming from the treatment head, even when wearing Lumenis eyewear. A recent Lumenis patent application states: “using energy sources . . . in the vicinity of the eye, such as the eyelids and adjacent regions of the face, also referred to as ocular and periocular/circumocular areas, may raise safety concerns.” See US 2013/0172959, ¶ 0001. Even now, IPL typically used by dermatologists is viewed as a real danger, as discussed in an Apr. 25, 2012 article in the US Edition of the Ocular Surgery News. When safety goggles were removed to treat difficult-to-reach periocular areas, two patients suffered permanent damage and pain from IPL. This article can be found on the Internet at http://www.healio.com/ophthalmology/cornea-external-disease/news/print/ocular-surgery-news/%7B958f87c5-d7dd-465e-bb17-bc3584240a08%7D/intense-pulsed-light-may-cause-damage-to-periocular-area.

Some effort has been made to solve the problem of changing eye color. U.S. Pat. No. 8,206,379 to Homer (“the '379 patent”) describes using a laser to remove iris pigment. This device has apparently been created and tested by Stroma Medical Corp. According to the Stroma website and articles it cites, the device used to implement the '379 patent is a Q-switched neodymium YAG laser, which produces a single absorbable wavelength. See also '379 patent, col. 3, lines 7-9; col. 7, lines 15, 22-32; col. 8, lines 55-58, discussing laser-generated single wavelengths. The Nd:YAG laser fires a large number of tiny, computer-guided pulses across the iris, to photodisrupt the stromal melanocytes, i.e., the cells that manufacture brown pigment, thus leaving a blue iris. This technique has certain limitations, because the laser makes holes in the iris, which can result in significant pain to patients. See '379 patent, col. 6, lines 36-40. It is impossible to numb the iris to the laser energy pulses, because topical numbing drops, such as those used in LASIK surgery, do not numb the inside of the eye, which is where the iris is located. The '379 patent offers no meaningful suggestion of using IPL to alter eye color, nor how such use could be implemented. The current view in the art is that IPL should not be used on or near the eye.

Therefore, despite the vague, speculative concepts put forth in the '379 patent, it is desirable to have a less painful, less costly, and more efficient way of changing eye color.

### SUMMARY OF THE INVENTION

The solution to this problem is the use of multichromatic IPL. Because of the photoabsorption properties of IPL, the energy passes through the clear cornea and strikes brown melanocytes in the iris. The cornea and the posterior iris stroma are unaffected as the eye pigment is destroyed. It should be noted that the melanin in the eye is a huge molecule and quite similar in biochemical, structural, and functional aspects to its equivalent found in skin and hair. Therefore, the ocular melanin will respond much like the melanin in the skin that is treated with IPL. Monochromaticity, i.e., a single pulsed wavelength from a laser, is not a prerequisite for selective heating of target structures in the body or the eye. The broad wavelength capability of an IPL device can generate simultaneous emission of multiple colors, i.e., a broad wavelength spectrum even including infrared light, because the entire spectrum to which melanin is susceptible can be generated by IPL. Modern IPL skin devices can provide pulse durations of 100 milliseconds and more, greater than that of a typical laser. This pulse time has the potential to provide small amounts of energy over longer periods of time than a laser, resulting in slow and gentle heating. Conversely, longer delays between pulses would allow the iris and the eye to cool, thus preventing damage from overheating. Another advantage of IPL is the relatively large footprint of its spot size. This could limit the total number of pulses per procedure and afford swifter treatment for the patient. As a result, IPL represents a favorable cost and versatility improvement over a traditional single spectrum laser. When used with the eye, the invention uses much less energy than IPL for skin and therefore has less risk to cause the damage that has resulted from accidental exposure during dermatological procedures.

The present invention includes a slit lamp microscope, a source of IPL, and an optical tracking and measuring system. Some form of protection for the pupil, whether mechanical or electronic, is also necessary. Control of the IPL requires a tracking device to follow the motion of the eye, and it requires a measurement system that controls the depth, timing, and spacing of each IPL application. As noted previously, some eye protection is necessary, because it is undesirable to expose the pupil to such light, and also desirable to limit the exposure of the sclera. The simplest means of protection would be a contact lens with a central opacity to cover and protect the pupil and prevent IPL from inadvertently entering the eye and exposing the retina to IPL. The preferred embodiment of the invention uses eye tracking and measuring systems to apply an annular ring of IPL just on the iris and not on the pupil or sclera, so that the opaque lens is unnecessary. The eye tracking mechanism would allow mirrors to follow any patient eye movements so IPL application to solely the iris tissue would be achieved and would avoid exposure to the pupil or sclera. In an abundance of caution, one could use both the mechanical contact lens and the optical tracking and measuring systems. This would additionally insure safe treatment of the eye.

In another embodiment, cooling devices that prevent the iris from overheating enhance safety further. Fans or air nozzles blow air toward the eye. Similarly, a liquid coolant could be sprayed or misted directly onto the eye.

### BRIEF DESCRIPTION OF THE DRAWINGS

Below is a detailed description that refers to the novel aspects of the invention, including equivalents known by those of skill in the art, and in that context refers to the following figures.

FIG. 1 depicts an eye, including anterior aspects such as the cornea, the pupil, the iris, the lens, and the sclera.

FIG. 2 is a general depiction of a prior art slit lamp microscope.

FIG. 3 is schematic drawing of one embodiment of the invention.

### DETAILED DESCRIPTION OF THE INVENTION

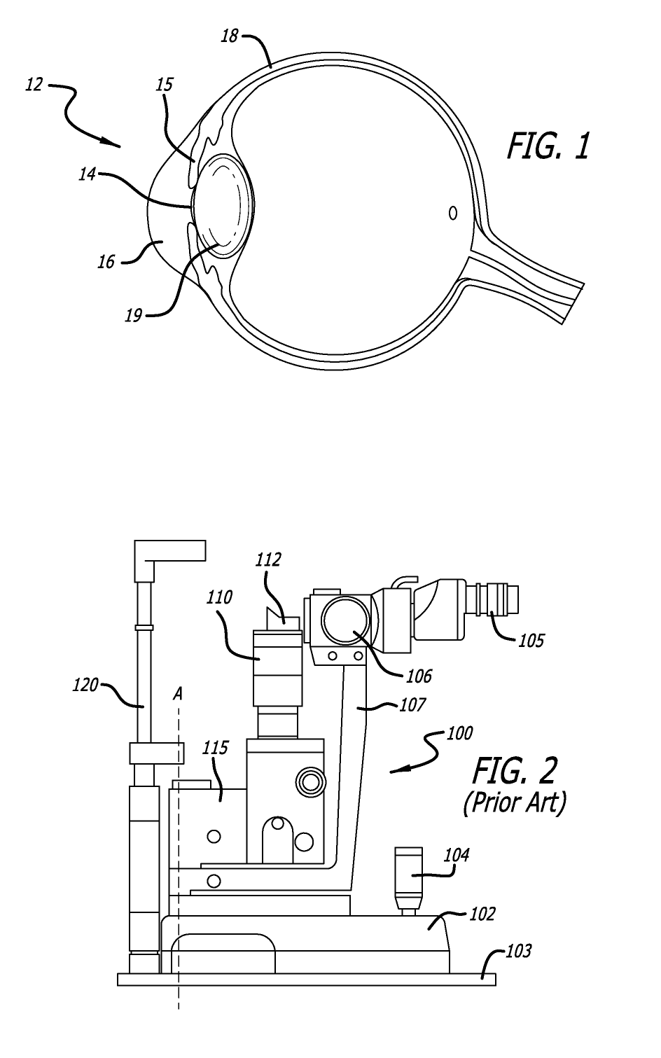

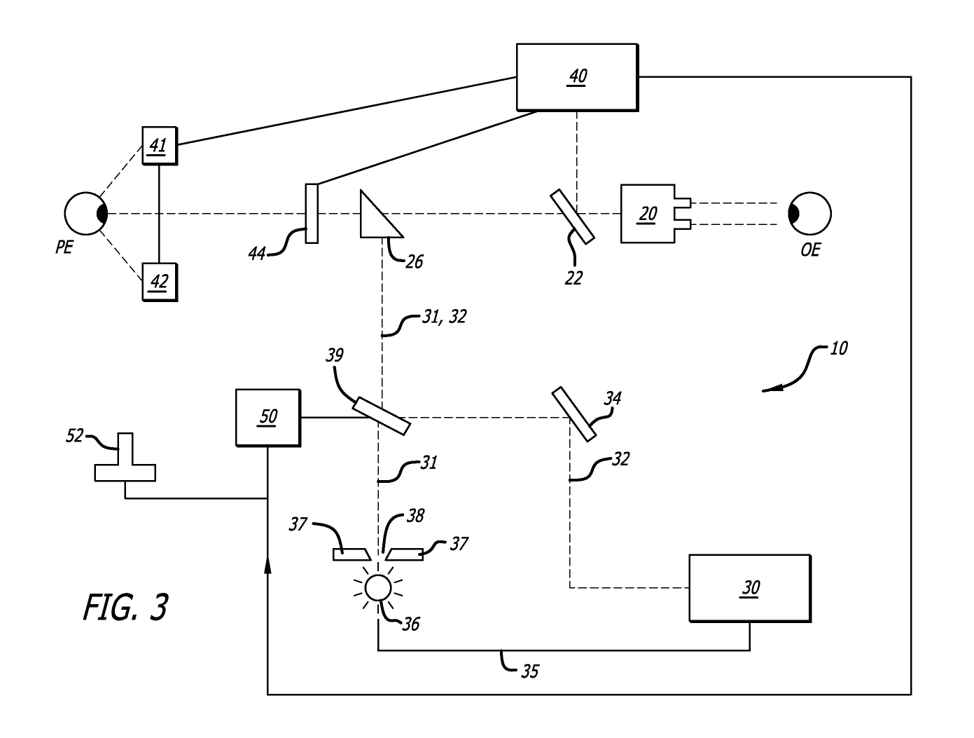

The purpose of the invention 10 is to remove pigment from the iris 15 of eye 12, without exposing pupil 14 to any harmful light. See FIG. 1 for a general depiction of the eye. One embodiment of the invention 10 includes a slit lamp microscope 20 that uses IPL 32 to remove iris pigment from a patient's eye PE. See FIG. 3. The IPL is applied multichromatically, as a diffuse light source, to heat the melanin. The preferable spectrum runs between about 500 and 1400 nm, although wavelengths as low as 250 nm have been used. Wavelengths can be adjusted lower or higher to account for variations in the melanin. Filters can be used to control the minimum and maximum wavelengths that are used in a single pulse or in multiple pulses. Depending on the nature of the patient's eye, and the amount of IPL used, eye color can be changed from dark (e.g., brown) to light (e.g., blue, green, hazel). It should be noted that this invention would not perform the reverse function, i.e., creating melanin to darken the eye.

Slit lamp microscope 20 includes several parts, such as binocular or monocular viewing for the operator's eyes OE, slit lamp 36, and slit plate 37 with slit 38, and joystick 52. Too much movement on the part of the patient might lead to an undesirable result, so for a procedure such as exposing the iris to IPL the patient's head ideally should be mechanically secured against movement, whereas a simple examination with a slit lamp microscope usually requires the patient to place his chin and forehead against the frame and avoid moving. More effective prevention of head movement can be accomplished by strapping or otherwise securing the patient's head to the frame that makes up the chin support and head support (see headrest 120, FIG. 2, strap not shown). Light and power source 30 provides power to slit lamp 36 through wire 35. Lamp 36 generates illuminating light 31, which travels to patient eye PE and reflects back to the operator's eyes OE looking through microscope 20. A joystick 52 controls the direction of the illuminating light 31 onto patient eye PE. As noted above, the slit lamp microscope may have other control features as well, which are known to those of skill in the art.

Light and power source 30 also contains the source that produces IPL 32, which reflects off mirror 34 onto waveform combining mirror 39, combining the illuminating light 31 with IPL 32. Light 31, 32 is reflected through prism mirror 26 to patient eye PE. Obviously the slit lamp 36 can be operated independently of IPL 32, so that the operator can examine the eye without applying IPL 32. Similarly, invention 10 contemplates using slit lamp 36 and IPL 32 concurrently, so that the operator can view the iris while IPL is applied during the treatment. While the operator's eyes OE are viewing the patient eye PE through microscope 20, beam splitter 22 creates two beams, each containing the illuminating light 31 and the IPL 32 reflected by the patient's eye PE. One beam goes to microscope 20 for viewing by the operator's eyes OE. It should be noted that the oculars of the microscope contain filters to prevent IPL treating the operator's irises and the operator would be required to wear protective eyewear for this purpose as well for additional operator protection. The other beam is diverted to the tracking and measuring systems 40 to provide continual feedback and control of the IPL application. Tracking and measuring systems 40 may also use a laser (not shown).

Eye tracking and measuring systems 40 control the depth, wavelength, timing, and spacing of each IPL application. Tracking and measuring the eye involve complex hardware, software, and optical systems that are known to those in the art of optical equipment design. In this simplified view, as shown in FIG. 3, trackers 41, 42 use infrared measurements as one embodiment of eye tracking to track the movement of patient eye PE. This information is fed back to the main tracking and measuring system 40 and to mirror control and IPL aiming system 50, which in turn aims IPL 32 through the control of mirrors 26, 39 and to patient eye PE. These mirrors are intended to generally represent the more complex and detailed aspects of aiming narrow light beams that are known to those of ordinary skill in the art. They can, for example, work with or independently of joystick 52. The type and degree of viewing control may be chosen by one of ordinary skill whose is designing the device.

Other necessary aspects of the invention are known to those of ordinary skill in the art, and thus need not be described here in detail. For example, virtually all commercially available lasers used in refractive surgery are associated with some form of eye-tracking system that can account for movement of both the eye and the head. Examples of prior art tracking systems include U.S. Pat. No. 6,280,436 to Freeman et al and US2002/0051116 to Van Saarloos et al. A tracker consists of a reception system and a repositioning system that maintains the laser within a specific tracking range. A passive system determines an interruption in the emission of pulses because of eye movements that exceed the tracking range, thus stopping the laser or other light source. In contrast, an active tracking system follows the ocular movements by centering the treatment on the exact position programmed at the start of surgery. Some tracking systems contain elements of both passive and active tracking.

In the preferred embodiment of the invention, the tracking system is combined with a measuring system that determines a variety of parameters necessary for the color change procedure, such as the geometry of the various parts of the eye including the cornea, the pupil, and the iris. Those of skill in the art are familiar with such systems, including aspects such as programming functions, hardware, software, and algorithms. Likewise, programmable and built-in features would permit the use of lightwave filters, IPL pulse length and spacing, the number of pulses, and other necessary features.

Cooling devices could also be added to prevent the iris from overheating. Fans or air nozzles could blow air directed toward the eye. Likewise, a liquid coolant could be sprayed or misted directly onto the eye. Such devices would be located near the patient's head. If the invention were constructed similar to the slit lamp microscope depicted in FIG. 2, one could attach cooling devices to headrest 120 or on separate structures located nearby.

Although the inventor has described what he considers the best mode of carrying out the invention, it will be apparent to those skilled in the art that modifications, variations, and equivalents can be made without departing from the scope of the invention as detailed in the claims below. For example, if it is desirable to heat chromophores other than melanin, like water, other wavelength ranges of IPL could be used. Those of skill in the art will appreciate that the physical form of the invention can vary. A single device can incorporate all of the elements of the invention. Alternatively, the invention can be a system of two or more separate elements linked together. Thus, one might refer to the invention as a device or as a system. A small example is the application of light. As described above, illumination light 31 and IPL 32 are preferably delivered together through combining mirror 39 and prism mirror 26. This physical arrangement, however, is not a requirement of the invention.

I claim:

1. A system for changing the color of the iris of an eye, comprising:
a slit lamp microscope;
a source of IPL for application to the iris and sufficient to cause the iris to become lighter in color, the IPL having a defined range of wavelengths;
a measuring and tracking system to control the application of the IPL to the iris; and,
means for preventing application of IPL to the pupil of the eye.

2. The invention of claim 1, wherein the means for preventing application includes at least one of an opaque cover of the pupil and a control of the measuring and tracking system that prevents application of the IPL to ocular structures other than the iris.

3. The invention of claim 2, wherein the control of the measuring and tracking system applies a generally annular-shaped ring of light only to the iris.

4. The invention of claim 2, wherein the control of the measuring and tracking system applies light in at least one of a triangle, rectangle, and a plurality of overlapping circles.

5. The invention of claim 2, wherein the IPL wavelengths are in a range of 250 to 700 nanometers.

6. The invention of claim 2, wherein the IPL wavelengths are in a range of 700 to 1,400 nanometers.

7. The invention of claim 2, further comprising an eye coolant applicator for applying at least one of an air and a liquid coolant.

8. The invention of claim 4, wherein the control of the measuring and tracking system applies a generally annular-shaped ring of light only to the iris.
